(19) United States
(12) Patent Application Publication
(10) Pub. No.: US 2008/0182887 A1
(43) Pub. Date: Jul. 31, 2008

(54) Stable Oral Pharmaceutical Composition

(75) Inventors: Vaishali Vijay Dhavse, Mumbai (IN); Nitin Bhalachandra Dharmadhikari, Mumbai (IN)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: SUN PHARMACEUTICAL INDUSTRIES LIMITED, Mumbai (IN)

(21) Appl. No.: 11/816,882

(22) PCT Filed: Feb. 22, 2006
(86) PCT No.: PCT/IN2006/000060

§ 371 Date: Aug. 22, 2007

(30) Foreign Application Priority Data
Feb. 22, 2005 (IN) 200/MUM/2005

(51) Int. Cl. A61K 31/40; A61P 7/00

(52) U.S. Cl. 514/423

(57) ABSTRACT

The invention relates to a stable oral pharmaceutical composition comprising atorvastatin or its pharmaceutically acceptable salt and a pharmaceutically acceptable carrier comprising tromethamine and an antioxidant.

The present invention provides a stable oral pharmaceutical composition comprising atorvastatin or its pharmaceutically acceptable salt and tromethamine in an amount sufficient to prevent degradation of the atorvastatin or its pharmaceutically acceptable salt.

### BACKGROUND OF THE INVENTION

Atorvastatin calcium, an HMG-CoA reductase inhibitor, disclosed in the U.S. Pat. No. 5,273,995, is currently sold in US as Lipitor® having chemical name [R-(R*,R*0]-2-(4-flurophenyl)-β,β-dihydroxy-5-(1-methylethyl)-3-phenyl-4-[(phenylamino)carbonyl]-1H-pyrrole-1-heptanoic acid calcium salt (2:1) trihydrate. It is susceptible to a low pH environment and can degrade to the corresponding lactone in an acidic environment. Mills et al have stated in U.S. Pat. No. 5,686,104 that this and similar compounds in an oral pharmaceutical composition for the treatment of hypercholesterolemia or hyperlipidemia are stabilized by combination with at least one basic inorganic pharmaceutically acceptable calcium, magnesium, aluminium or lithium salt. Examples of these salts are calcium carbonate, calcium hydroxide, magnesium carbonate, magnesium hydroxide, magnesium silicate, magnesium aluminate, aluminium hydroxide or lithium hydroxide.

WO00/35425 ('425 application) discloses the stabilization of an HMG-CoA reductase inhibitor in a solid formulation with a buffering agent. Among the buffering agents disclosed in '425 application are sodium or potassium citrate, sodium phosphate, sodium sulphate, sodium or magnesium carbonate, sodium ascorbinate, sodium benzoate, sodium or potassium hydrogen carbonate, lauryl sulphate, or mixtures of such buffering agents,

WO01/76566 discloses a stabilized pharmaceutical composition comprising at least one ring-opened 7-substituted-3,5-dihydroxyheptanoic acid or ring-opened 7-substituted-3,5-dihydroxyheptenoic acid, or a pharmaceutically acceptable acid salt thereof, and a stabilizing effective amount of at least one amido-group containing polymeric compound or at least one amino-group containing polymeric compound or combination thereof, wherein said stabilized pharmaceutical composition does not contain a stabilizing effective amount of another stabilizer or a combination of other stabilizers. These amido-group containing polymeric compounds were selected from the group consisting of polyvinylpyrrolidone, cross-linked polyvinylpyrrolidone, copolymers of vinylpyrrolidone and vinyl acetate and polynoxylin.

WO2004/071402 relates to stable pharmaceutical dosage forms comprising the HMG-CoA reductase inhibitors and pharmaceutical excipients that do not contain alkalizing or buffering substances or combinations thereof.

US20040247673 discloses a wet granulated pharmaceutical composition of atorvastatin with less than 5 weight % of an alkaline earth metal salt additive comprising: a) atorvastatin or a pharmaceutically acceptable salt thereof; and b) a disintegrant or combination of disintegrants, wherein said wet granulated pharmaceutical composition contains not more than about 3% atorvastatin lactone based on the ratio of lactone peak area compared to the total drug-related peak integrated areas using HPLC. The patent teaches that certain disintegrants may be detrimental to the stability of atorvastatin for example, croscarmellose, Tween in amounts more than 0.5% w/w. Moreover, the patent states that because the alkaline earth metal salt additives can affect atorvastatin bioavailability, it is needed to provide atorvastatin in a wet granulated composition substantially free of alkaline earth metal salt additive.

U.S. patent application No. 20040138290 relates to a pharmaceutical formulation containing an alkalizing agent or a buffering substance which improves the bioavailability of atorvastatin by increasing its solubility and dissolution rate in aqueous solutions. The examples of this invention use high amounts, i.e more than 25% of tromethamine to get the desired pH. The patent application does not mention the stability of the composition with respect to its lactone content.

U.S. Pat. No. 6,806,290 discloses a composition comprising a homogeneous mixture of a HMGCoA reductase inhibitor with a buffering substance or a basifying substance in a finely distributed form obtained by co-crystallization and/or co-precipitation of said HMGCo A reductase inhibitor and said buffering substance or basifying substance, which has a HPLC purity of at least 98%. The basifying substances exemplified in the patent are Tris (hydroxymethyl) amino ethane, N,N′-bis (2-hydroxyethyl) ethylene diamine.

We have surprisingly found a stable oral pharmaceutical composition comprising atorvastatin or its pharmaceutically acceptable salt and tromethamine in an amount sufficient to prevent degradation of the atorvastatin or its pharmaceutically acceptable salt.

### OBJECTS OF THE INVENTION

It is the object of the present invention to provide a stable oral pharmaceutical composition comprising atorvastatin or its pharmaceutically acceptable salt.

It is a further object of the present invention to provide a stable oral pharmaceutical composition comprising atorvastatin or its pharmaceutically acceptable salt and tromethamine and an additional stabilize in an amount sufficient to prevent degradation of the atorvastatin to less than 0.5% of lactone by weight of the atorvastatin or its pharmaceutically acceptable salt.

It is further object of the invention to provide a stable oral pharmaceutical composition comprising atorvastatin or its pharmaceutically acceptable salt and a pharmaceutically acceptable carrier comprising a stabilizer and containing atorvastatin lactone less than 0.5% by weight of atorvastatin wherein the amount of atorvastatin lactone remains substantially unchanged upon storage at 40° C. and 75% relative humidity in a closed container containing a dessicant for a period of three months.

Another object of the invention is to provide a stable oral pharmaceutical composition comprising atorvastatin or its pharmaceutically acceptable salt and pharmaceutically acceptable carrier comprising about 0.5% to about 3.0% by weight of tromethamine and an additional stabilizer in an amount sufficient to prevent degradation of atorvastatin or its pharmaceutically acceptable salt wherein the composition is devoid of a disintegrant.

### SUMMARY OF THE INVENTION

The present invention provides a stable oral pharmaceutical composition comprising atorvastatin or its pharmaceutically acceptable salt and pharmaceutically acceptable carrier comprising about 0.5% to about 3% by weight of tromethamine and an additional stabilizer in an amount sufficient to prevent degradation of atorvastatin or its pharmaceutically acceptable salt.

The present invention particularly provides a stable oral pharmaceutical composition comprising calcium salt of atorvastatin.

Further the present invention provides a stable oral pharmaceutical composition comprising atorvastatin or its pharmaceutically acceptable salt and pharmaceutically acceptable carrier comprising about 0.5% to about 3% by weight of tromethamine and additional stabilizer in an amount sufficient to prevent degradation of atorvastatin or its pharmaceutically acceptable salt to less than 0.5% by weight of lactone.

Also the present invention provides a stable oral pharmaceutical composition comprising atorvastatin or its pharmaceutically acceptable salt and pharmaceutically acceptable carrier comprising a stabilizer and containing atorvastatin lactone less than 0.5% by weight of atorvastatin wherein the amount of atorvastatin lactone remains substantially unchanged upon storage at 40° C. and 75% relative humidity in a closed container containing a dessicant for a period of 3 months.

Another object of the invention is to provide a stable oral pharmaceutical composition comprising atorvastatin or its pharmaceutically acceptable salt and pharmaceutically acceptable carrier comprising about 0.5% to about 3.0% by weight of tromethamine and additional stabilizer in an amount sufficient to prevent degradation of atorvastatin or its pharmaceutically acceptable salt wherein the composition is devoid of a disintegrant.

### DETAILED DESCRIPTION OF THE INVENTION

It has been surprisingly found that a stable oral pharmaceutical composition comprising atorvastatin or its pharmaceutically acceptable salt can be obtained by providing, in a pharmaceutical composition, a stabilizing effective amount of tromethamine and an additional stabilizer.

The term “stable” indicates that when the composition is stored at 40° C. at 75% relative humidity (RH) in closed containers with dessicant for extended period of time such as three months, the corresponding lactone formation does not exceed 0.5% by weight to the atorvastatin or its pharmaceutically acceptable salt.

The term “lactone content remains substantially unchanged” means that when the composition of the present invention is stored at 40° C. and 75% relative humidity in closed containers having a dessicant, the analytical values of lactone content does not change by more than 30% from the initial value.

The lactone formation was assessed by testing the compositions of the present invention, which are stored in HDPE plastic bottles with silica gel as the desiccant at 40° C. at 75% RH (at 40° C.±2° C., 75% RH±5%). The assay of degradation products of atorvastatin, i.e lactone, was determined by HPLC (Waters Symmetry, C-8, 150×4.6 mm, particle size 5 μm, thermostated to 25° C.; injection volume: 20 μL, flow rate: 2.0 ml per minute; 254 nm detection). The elution used mobile phase of citrate buffer: acetonitrile: Tetrahydrofuran (57:23:20). Lactone content was calculated on the basis of a standard curve derived from a pure atorvastatin lactone standard. The lactone impurity, was reported as percentage w/w with respect to the atorvastatin content of the pharmaceutical composition.

The present invention is particularly adapted to stable oral pharmaceutical compositions comprising atorvastatin or its pharmaceutically acceptable salt, as the active component of the composition. Among the preferred pharmaceutically acceptable salts are metal and amine salts. The term “pharmaceutically acceptable salt” thus includes, but is not limited to sodium, potassium, lithium, calcium, magnesium, aluminium, iron and zinc salts of atorvastatin. Preferably, the atorvastatin is in the form of calcium salt.

The atorvastatin or its pharmaceutically acceptable salt is used in a therapeutically effective amount in the stable oral pharmaceutical compositions of the present invention. The atorvastatin or its pharmaceutically acceptable salt will generally be present in an amount within the range of from about 0.05 to about 70%, and preferably an amount within the range of from about 1 to about 60% by weight of the composition, most preferably from about 5% to about 40% by weight of the composition. The therapeutic effective amount of atorvastatin or its pharmaceutically acceptable salt that may be used in the stable composition of the present invention is in the range from about 10 to about 80 mg, equivalent to the base.

Also present in the stable oral pharmaceutical composition of the present invention is a stabilizing effective amount of tromethamine. Tromethamine, a non-volatile primary amine, is known by various names, for example, tris (hydroxymethyl) amino methane, tromethane, Addex-Tham, 1,1,1-tris (hydroxymethyl_-methanamine, Pehanorm, Talatrol, Tham-E, trimethylolaminomethane, tris (hydroxymethyl) methylamine, Tris-steril, Tris free base, Trisamin, Trisamine, Trisaminol, Trisbuffer, Trizma, tromethane, trometamol, Apiroserum and among others.

The amount of tromethamine that may be used in the stable oral pharmaceutical composition of the subject invention varies from about 0.5 to about 3.0% w/w of the composition, more preferably, from about 1% to about 2% w/w of the total weight of the composition, most preferably less than 1% w/w of the composition.

Improved stability may be achieved by incorporating additional stabilizer. The additional stabilizer that may be used in the present invention includes antioxidants selected from the group comprising butylated hydroxy anisole, butylated hydroxy toluene, DL-alpha-tocopherol, propyl gallate, octyl gallate, ethylenediamine tetraacetate, ascorbyl palmitate, acetyl cysteine, ascorbic acid, sodium ascorbate, fumaric acid, lecithin and the like and mixtures thereof. The amounts of the antioxidants may vary from about 0.001% to about 0.01% w/w of the composition, preferably from about 0.005 to about 0.01%, most preferably 0.009% w/w of the composition.

Another stabilizer that may be used in addition to tromethamine is sodium lauryl sulphate. The amounts of sodium lauryl sulphate may range from about 1% to about 5% by weight of the composition, preferably less than 3% and most preferably less than 2% by weight of the composition.

In more preferred embodiments of the present invention the stabilizer comprises tromethamine, antioxidants and sodium lauryl sulphate.

The pharmaceutical composition of the present invention further comprises conventional pharmaceutically acceptable excipients that form the pharmaceutical carrier. Conventional pharmaceutical excipients include those which function in a dosage form, for example, as a lubricant, glidant, fillers, wicking agents, carrier, colorant or coating material.

The pharmaceutical composition of the present invention preferably does not contain a disintegrant. Certain disintegrants are deleterious to the stability of atorvastatin. The pharmaceutical composition of the present invention comprises in addition to the stabilizers, additional pharmaceutically acceptable excipients selected from fillers, binders, lubricants, glidants, wicking agents, sweetening agents, wetting agents, flavouring agents, coloring agents and other such excipients.

In preferred embodiments the composition of the present invention is formed into tablets. The fillers and binders that are used enable the disintegration of the tablet without the need of a disintegrant. Fillers that may be used in the stable oral pharmaceutical composition of the present invention microcrystalline cellulose, mannitol, dextrates, dextrins, dextrose, fructose, lactose, lactitol, maltitol, maltodextrins, maltose and the like. Preferably the filler is microcrystalline cellulose. The commercially available grades of microcrystalline cellulose that may be used are Avicel PH-102 and Avicel PH-112, both have a mean particle size of 100 μm with moisture content being less than 5.0% and less than 1.5% respectively.

The binders used in the present invention may be selected from the group comprising of starch, gelatin, dextrin, maltodextrin, natural and synthetic gums like acacia, alginic acid, sodium alginate, guar gum, extract of Irish moss, ghatti gum, mucilage of isapol husks, carboxymethylcellulose, methylcellulose, hydroxyethyl cellulose, hydroxypropyl cellulose, hydroxypropyl methylcellulose, polyvinylpyrrolidone, veegum, arabogalactan and the like and mixtures thereof. Generally the amount of the binders used in the present invention may vary from about 0.5% w/w to about 10% w/w of the composition.

The lubricants used in the present invention may be selected from the group consisting of talc, magnesium stearate, calcium stearate, stearic acid, hydrogenated vegetable oils, polyethylene glycol and the like and mixtures thereof. Generally the amount of the lubricants used in the present invention may vary from about 0.001 to about 5% w/w of the composition.

The typical glidants that may be included in the present invention include colloidal silicon dioxide, talc and the like. The amounts of glidants used in the present invention may vary from about 0.1 to about 5% w/w of the composition.

Examples of wicking agents that may be used in the present invention include colloidal silicon dioxide, kaolin, titanium dioxide, fumed silicon dioxide, m-pyrol, vinylpyrrolidone polymers such as povidone, or crosslinked polyvinylpyrrolidone such as crospovidone; cellulose and cellulose derivatives such as microcrystalline cellulose, methylcellulose, ethylcellulose, hydroxypropylcellulose, hydroxypropyl cellulose, carboxyallcyl celluloses and their alkali salts; sodium starch glycolate, starch and starch derivatives, ion-exchange resins and the like and mixtures thereof.

The term “pharmaceutical composition” as used herein includes solid oral dosage forms such as pellets, beads, granules and the like, which may be encapsulated or compressed into tablets. The pellets, beads, granules in turn may be prepared by conventional methods known to a person skilled in the art. The compressed tablets may optionally be coated with film-coat.

Preferred embodiments of the present invention are stable when stored at 40° C. at 75% RH for 3 months and contain less than 0.5% w/w atorvastatin lactone after such storage. In more preferred embodiments of the present invention the amount of atorvastatin lactone remains substantially unchanged under these conditions.

The pharmaceutical compositions of the present invention, may be prepared by the conventional processes such as wet granulation, dry granulation or direct compression. In wet granulation, atorvastatin or its pharmaceutically acceptable salt is mixed with tromethamine and various excipients and granulated, followed by screening and drying of the damp mass. The dried mass may be screened, lubricated and compressed. Dry granulation can be done by two processes: (1) slugging, which involves mixing the atorvastatin or its pharmaceutically acceptable salt with tromethamine and the excipients, slugging, dry screening, lubrication and compression, or (2) roller compaction process. Direct compression involves compressing tablets directly from the physical mixture of atorvastatin or its pharmaceutically acceptable salt, tromethamine and the excipients.

Alternatively the pharmaceutical compositions of the present invention may be obtained by preparing placebo granules comprising the tromethamine and pharmaceutically acceptable excipients, and mixing these with atorvastatin to obtain a blend, which may be encapsulated or compressed into tablets. This method provides compositions of atorvastatin that are stable.

The examples that follow do not limit the scope of the invention and are merely used as illustrations.

### EXAMPLE I-V

An oral composition comprising atorvastatin or its pharmaceutical acceptable salt were obtained as described in Table 1 below.

TABLE 1

| | Formula (% w/w of ingredients) | | | | |
|---|---|---|---|---|---|
| Name of the Ingredient | I | II | III | IV | V |
| Atorvastatin Calcium | 14.03 | 7.01 | 8.42 | 8.42 | 8.42 |
| Lactose anhydrous | 19.41 | — | — | — | — |
| Microcrystalline cellulose (Avicel PH 112) | 36.2 | 51.8 | 51.9 | 52.9 | 51.9 |
| mannitol DC grade (Pearlitol SD 200) | — | 25.8 | 26.4 | 26.4 | 28.1 |
| L-hydroxypropylcellulose (L-HPC) | 8.09 | 4.04 | 3.9 | 3.9 | 3.9 |
| Tromethamine | 12.94 | 3.88 | 1.94 | 0.97 | 1.94 |
| Sodium lauryl sulphate | 3.88 | 1.94 | 1.94 | 1.94 | — |
| Tween 80 | — | — | — | — | 0.38 |
| Butylated hydroxy toluene (BHT) | 0.009 | 0.009 | 0.009 | 0.009 | 0.009 |
| Butylated hydroxy Anisole (BHT) | 0.009 | 0.009 | 0.009 | 0.009 | 0.009 |
| Colloidal silicon dioxide | 1.45 | 1.45 | 1.45 | 1.45 | 1.45 |
| Magnesium stearate | 0.97 | 0.97 | 0.97 | 0.97 | 0.97 |
| Opadry White | 2.91 | 2.91 | 2.91 | 2.91 | 2.91 |

BHA and BHT were dissolved in Isopropyl alcohol. Atorvastatin calcium, L-HPC (LH-11) and Tromethamine were dry mixed. The solution of BHA, BHT was added to the dry mix followed by addition of mannitol, microcrystalline cellulose and optionally, sodium lauryl sulfate. This mixture was wet milled to obtain granules, which were dried to moisture content between 2.0 to 3.0%. The dried mass was lubricated with colloidal silicon dioxide and magnesium stearate and compressed into tablets. The tablets were coated with aqueous Opadry White 03B68903 to a weight gain of about 3% by weight of compressed tablet.

### COMPARATIVE EXAMPLE VI

TABLE 2

| | Formula VI |
|---|---|
| Name of the Ingredient | (% w/w of ingredients |
| Atorvastatin Calcium | 7.01 |
| Lactose momohydrate | 21.60 |
| Microcrystalline cellulose (Avicel PH 101) | 38.83 |
| Calcium carbonate | 21.35 |
| Croscarmellose | 5.82 |
| Butylated hydroxy Anisole (BHA) | 0.01 |
| Hydroxypropyl cellulose | 1.94 |
| Magnesium stearate | 0.48 |
| Opadry White | 2.91 |

The composition according to formula VI was prepared according to the teachings of U.S. Pat. No. 5,686,104 (US '104). Hydroxypropyl cellulose was dispersed in water.

BHA was dissolved in isopropyl alcohol. Atorvastatin calcium, lactose monohydrate, microcrystalline cellulose, croscarmellose sodium and calcium carbonate in the specified quantities were sifted and dry mixed. The dry mix was granulated with HPC and BHA in isopropyl alcohol. The granules were dried. Croscarmellose and magnesium stearate were sifted and blended with granules. These granules were lubricated with magnesium stearate and compressed into tablets. The compressed cores were coated with aqueous Opadry to a weight gain of about 3% by weight of the compressed tablet.

### EXAMPLE VII

The compositions prepared according to formulae I-VI were stored at 40° C. at 75% RH for 3 months in HDPE bottles with silica gel as desiccants. The lactone content was determined by HPLC and is recorded in table 3 below.

TABLE 3

% w/w lactone content at 40° C.
at 75% RH for three months

| | | 1 | 2 | 3 |
|---|---|---|---|---|
| Formula | Initial | month | month | month |
| I | 0.20 | — | — | 0.25 |
| II | 0.18 | 0.19 | 0.19 | 0.22 |
| III | 0.24 | 0.19 | 0.13 | 0.26 |
| IV | 0.09 | 0.02 | — | 0.18 |
| V | — | 0.02 | — | 0.15 |
| VI- | 0.23 | 0.30 | 0.36 | 0.59 |
| Comparative | | | | |

It is apparent from Table 3 above that use of tromethamine prevent degradation of atorvastatin or its pharmaceutically acceptable salt to the corresponding lactone and that tromethamine in combination with antioxidants provides superior protection against lactone formation as compared to prior art (US '104) comparative example VI. Further the example demonstrates that the compositions of present invention, which further use sodium lauryl sulphate, the lactone content remains substantially unchanged (i.e with analytical values of lactone content does not change by more than 30% from the initial value).

While the invention has been described by reference to specific embodiments, this was done for purposes of illustration only and should not be construed to limit the spirit or the scope of the invention.

1. A stable oral pharmaceutical composition comprising atorvastatin or its pharmaceutically acceptable salt and pharmaceutically acceptable carrier comprising about 0.5% to about 3.0% by weight of tromethamine and an additional stabilizer in an amount sufficient to prevent degradation of the atorvastatin or its pharmaceutically acceptable salt and containing atorvastatin lactone less than 0.5% by weight of atorvastatin wherein the amount of atorvastatin lactone remains substantially unchanged upon storage at 40° C. at 75% relative humidity for 3 months in closed container containing a dessicant.

2. A stable oral pharmaceutical composition as in claim 1 wherein said atorvastatin is the calcium salt of atorvastatin.

3. A stable oral composition as in claim 1, wherein the additional stabilizer is an antioxidant selected from a group comprising, butylated hydroxytoluene, butylated hydroxyanisole, DL-alpha-tocopherol, propyl gallate, octyl gallate, ethylenediamine tetraacetate, ascorbyl palmitate, acetyl cysteine, ascorbic acid, sodium ascorbate, fumaric acid, lecithin and mixtures thereof.

4. A stable oral composition as in claim 3, wherein the amount of antioxidant ranges from about 0.001% to about 0.01% by weight of the composition.

5. A stable oral pharmaceutical composition as in claim 1 wherein the stabilizer is sodium lauryl sulphate.

6. A stable oral pharmaceutical composition as in claim 5 wherein the amount of sodium lauryl sulphate ranges from about 1 to about 5% by weight of the composition.

7. A stable oral pharmaceutical composition as in claim 1 containing less than 0.5% w/w atorvastatin lactone after storage at 40° C. at 75% relative humidity for 3 months in closed container containing a dessicant.

8. A stable oral pharmaceutical composition as in claim 1 wherein said composition is packed in closed containers containing a dessicant.

9. A stable oral pharmaceutical composition as in claim 7 wherein the atorvastatin lactone content remains substantially unchanged after storage at 40° C. at 75% relative humidity for 3 months in closed containers containing a dessicant.

10. A stable oral pharmaceutical composition as in claim 1 wherein the composition is devoid of a disintegrant.

11. (canceled)
